(19) United States
(12) Patent Application Publication
(10) Pub. No.: US 2016/0074209 A1
(43) Pub. Date: Mar. 17, 2016

(54) ELECTRIC THERMAL STIMULATION DEVICE AND ELECTRIC THERMAL STIMULATION CONTROL METHOD

(71) Applicant: Kenji RYOTOKUJI, Chiba-shi (JP)

(72) Inventor: Kenji RYOTOKUJI, Chiba-shi (JP)

(21) Appl. No.: 14/379,927

(22) PCT Filed: Sep. 12, 2012
(86) PCT No.: PCT/JP2012/073972

§ 371 Date: Dec. 1, 2015

(30) Foreign Application Priority Data
Feb. 24, 2012 (JP) 2012-038390
Feb. 24, 2012 (JP) 2012-038395

(51) Int. Cl. A61F 7/00

(52) U.S. Cl. CPC A61F 7/007; A61F 2007/0045; A61F 2007/0047; A61F 2007/0093

(57) ABSTRACT

This electric thermal stimulation device measures, with a laser Doppler tissue blood flowmeter worn on the center of the inside of the wrist joint, blood flow to the body surface before and after stimulation to a specific stimulation site. The blood flow amount supplied to the stimulated site is controlled to be 60% or greater, and preferrably 100% or greater

### TECHNICAL FIELD

The present invention relates to an electrical thermal stimulus apparatus and method for controlling electrical thermal stimulus apparatus that activate peripheral circulation as well as autonomic nervous functions by thermally stimulating a stimulus application site on a body surface.

### BACKGROUND ART

Various an electrical thermal stimulus apparatus are conventionally known. For example, an electrical thermal stimulus apparatus heats an acupuncture point as the tip of a heater element is affixed to the acupuncture point and then a push-type switch is turned on for energization (see Patent Literature 1). Additionally, an an electrical thermal stimulus apparatus controls energization to a thermotherapeutic device with continuous heating for a fixed period (see Patent Literature 2), and rapidly heats up to a predetermined temperature and then immediately rapidly radiates heat (see Patent Literature 2).

PTL 1

Japanese Registered Utility Model No. 3047096

PTL 2

Japanese Unexamined Patent Application Publication No. 05-277194

PTL 3

Japanese Unexamined Patent Application Publication No. 2004-173750

### SUMMARY OF INVENTION

### Technical Problem

The apparatus in Patent Literature 1 cannot provide thermal stimulus continuously. In the heating method in Patent Literature 2, it takes time to heat up to a target temperature, and is likely to cause a lower temperature burn as heating is continued further.

In the heating method in Patent Literature 2, it is difficult to control a temperature for sharply increasing a temperature and then quickly radiating heat and to maintain a desired temperature profile continuously. Thus, with the method, effective thermal stimulus cannot be provided.

An object of the present invention is to provide an electrical thermal stimulus apparatus and a method for controlling an electrical thermal stimulus apparatus for effectively stimulating to a thermal stimulus applied site on a body surface. A inventor found a new perspective in that the rate of increase in blood flow is an indicator for stimulus application after repeated extensive studies. Herein in the an electrical thermal stimulus apparatus and the method for controlling an electrical thermal stimulus apparatus, thermal stimulus is applied to a thermal stimulus applied site on a body surface, and the rate of increase in blood flow before and after the stimulus application is measured with a laser Doppler tissue blood flowmeter mounted at the center of an inside wrist joint.

### Solution to Problem

The an electrical thermal stimulus apparatus comprises a thermal stimulus controller that controls at least one of a heating temperature, a thermal stimulus waveform, a thermal stimulus intensity, a thermal stimulus applied time, a cycle of a thermal stimulus waveform, a thermal stimulus pattern, a warming mode and a thermal stimulus protocol,

wherein the heating temperature is controlled at 46.5° C. to 52.5° C. herein, and the thermal stimulus patterns are controlled independently by offsetting phases so as not to essentially overlap each other, and a blood flow before and after the application of thermal stimulus is measured with a laser Doppler tissue blood flowmeter mounted at the center of an inside wrist joint, and the rate of increase in blood flow after the application of thermal stimulus will be 60% or more, or preferably 100% or more, the rate of increase in blood flow is used as an indicator for the stimulus application.

An Electrical thermal stimulus apparatus comprises an electrical thermal stimulus controller, the electrical thermal stimulus controller controls non-simultaneously and independently applying a thermal stimulus with a peak temperature 50±5° C. by heating to thermal stimulus guide elements placed the different thermal stimulus applied sites of a body surface comprising the part that intersects the perpendicular line of the medial malleolus on extension line of the medial margin on os metatarsale primam 1 and 2 interosseous at foot of right and left by thermal stimulus applying apparatus to bring an increase rate of blood flow to 60% or more at before and after of the stimulus, wherein the blood flow is measured by a laser Doppler tissue blood flow meter attached to a central part of an inner side of a wrist joint and the increase rate of the blood flow is used as an indicator of a stimulus evaluation.,

In the an electrical thermal stimulus apparatus, the thermal stimulus controller controls a cycle of thermal stimulus pattern comprising the thermal stimulus waveform configured by the heating waveform and a thermal release waveform, and an interval to the following thermal stimulus waveform. And in one cycle of the thermal stimulus patterns, a thermal stimulus waveform is set at 1 to 30 seconds and the interval between the thermal stimulus waveform is set at 1 to 10 seconds.

In the electrical thermal stimulus apparatus the electrical thermal stimulus apparatus comprises the thermal stimulus guide element composed of two kinds of different metals.

In the method for controlling a thermal stimulus controller of an electrical thermal stimulus apparatus, the thermal stimulus controller controls at least one of a heating temperature, a thermal stimulus waveform, a thermal stimulus intensity, a thermal applied time, a cycle of a thermal stimulus waveform, a thermal stimulus pattern, a warming mode and a thermal stimulus protocol, wherein the heating temperature is controlled at 46.5° C. to 52.5° C. herein, and the thermal stimulus patterns are controlled independently by offsetting phases so as not to essentially overlap each other, and a blood flow before and after the application of thermal stimulus is measured with a laser Doppler tissue blood flowmeter mounted at the center of an inside wrist joint, and it is found that the rate of increase in blood flow after the application of thermal stimulus will be 60% or more, or preferably 100% or more, the rate of increase in blood flow is used as an indicator for the stimulus application.

In the method for controlling a thermal stimulus controller of an electrical thermal stimulus apparatus, the thermal stimulus controller controls a cycle of thermal stimulus pattern comprising the thermal stimulus waveform configured by the heating waveform and a thermal release waveform, and an interval to the following thermal stimulus waveform. And in one cycle of the thermal stimulus patterns, a thermal stimulus waveform is set at 1 to 30 seconds and the interval between the thermal stimulus waveform is set at 1 to 10 seconds.

And the thermal stimulus patterns are controlled independently by offsetting phases so as not to essentially overlap each other.

In the method for controlling a thermal stimulus controller of an electrical thermal stimulus apparatus, the thermal stimulus is applied the thermal stimulus applied site by thermal stimulus guide elements placed at a body surface comprising at least two of part of os metatarsale primam 1 and 2 interosseous, part os metatarsale primam and 3 interosseous, and part that intersects the perpendicular line of the medial malleolus on an extension line of the medial margin on os metatarsale primam 1 and 2 in foot sole of right and left.

### Advantageous Effects of Invention

As controlled thermal stimulus is effectively applied to a thermal stimulus applied site, blood flow increases.

Thermal Stimulus is applied to at least two locations at a thermal stimulus applied site non-simultaneously and independently from each other.

Accordingly, there provided were an increase in a deep body temperature, a decline of systolic blood pressure, a decrease in salivary amylase, a decrease in cortisol, an improvement of liver functions, a decrease in total cholesterol, a decline of good and bad cholesterol, a decline of glucose level, a decline of AI (arteriosclerotic index) and a decrease in leptin. Furthermore, the balance of an autonomic nerve was measured with a heart rater, which showed an increase in resistance against stress.

### BRIEF DESCRIPTION OF THE DRAWINGS

FIG. 1 a schematic view of an electrical thermal stimulus apparatus according to the invention.

FIG. 2 is a schematic view of a circuit diagram of the electrical thermal stimulus apparatus.

FIG. 3 shows a thermal stimulus waveform and cycle of a thermal stimulus waveform which is obtained by controlling a thermal stimulus apparatus.

FIG. 4 is a schematic view of a arrangement of function in the electrical thermal stimulus apparatus according to the invention.

FIG. 5 is a schematic view of a thermal stimulus applied site at sole.

FIG. 6 is a schematic view of a thermal stimulus protocol to a thermal stimulus applied site at sole

### DESCRIPTION OF EMBODIMENTS

The present inventor found that blood flow increases before and after application of thermal stimulus applied to a thermal stimulus applied site on a body surface with an electrical thermal stimulus apparatus. Based on the perspective that blood flow increases with the application of thermal stimulus to a thermal stimulus applied site on a body surface, an electrical thermal stimulus apparatus and the method for controlling electrical thermal stimulus apparatus provides for applying thermal stimulus to a stimulus applied site so that the rates of increase in blood flow will be at 60% or more, or preferably 100% or more. The rates of an increase in blood flow after stimulus obtained from the application of stimulus to a stimulus applied site is used as an indicator of thermal stimulus. Herein, the thermal stimulus applied site on a body surface is a site where the increase in blood flow is recognized by applying the thermal stimulus.

The thermal-stimulus origin parts of a body surface comprises at least two of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

Here, it is desirable to measure the volume of blood flow by the laser Doppler tissue rheometer which is attached to an inner and central part of a wrist joint. In a laser tissue blood flowmeter ALF21D (manufactured by ADOVANS), when a living tissue is irradiated with semiconductor laser light (whose wavelength is 780 mu), light reflected from the tissue is converted into an electric signal and the electric signal is processed, thereby obtaining the blood flow information. A C type laser probe (10 mm in diameter, 3 mm in thickness, 2 mm2 in a laser irradiation area, and 1 mm in measurement depth) of the laser tissue blood flowmeter ALF21D was attached to a central part of a wrist joint horizontal line of a healthy adult, and change of the blood flow volume was measured, taking a 15-minute rest after a stimulus.

As to the principle of the laser tissue blood flow volume measurement, laser light collides with red blood cells, which flow through the inside of blood vessels, and the Doppler shift (frequency change), which is produced when receiving dispersion, is used. This measurement method is characterized by non-invasive and real time responsiveness and a capability of consecutive measurement. Furthermore, the ALF21D, which was used this time, can display a blood flow volume on ml/min/100 g scale. This is because signal processing is performed based on the theory of Bonner et al.

The invention applies a thermal stimulus at a thermal stimulus applied site on body surface with an electrical thermal stimulus apparatus.

And the volume of blood flow was measured before and after the application of stimulus to a central part of a horizontal line of a wrist joint using a laser Doppler tissue blood flowmeter. And a thermal stimulus is applied at a thermal stimulus applied site on body surface so that an increase in blood flow volume is obtained. An increase in blood flow volume is used as indicator of an application of thermal stimulus. The increase rate of blood flow is desirably controlled to 60% or greater, preferably 100% or greater.

The thermal stimulus desirably includes non-simultaneously and independent thermal stimulus waveforms whose phases are shifted so that the patterns of thermal stimulus do not substantially overlap each other at a least different two thermal stimulus applied site. The thermal stimulus is desirably a thermal stimulus.

The thermal stimulus waveform includes a heating waveform obtained by heating it to a predetermined peak temperature, for example, 50±5 degrees Celsius, and a thermal release waveform which is formed by stopping heating after it reaches the peak temperature.

A cycle of the thermal stimulus waveform is desirably repeated continuously. Moreover, the thermal waveforms are not limited to the above-described thermal waveform. It may be a saw-toothed shape waveform, and concavo-convex waveform. Moreover, the heating waveform and the heat release waveform may be formed as a sine waveform.

One cycle pattern of the thermal stimulus is desirably set so that a thermal waveform time may be 1 0 second to 30 second and an interval time between may be 1 second to 10 seconds.

The thermal stimulus applied site is between the outer skin and a horizontal line of the inside boundary part of a hallux distal phalanx bottom and a proximal phalanx bone head in foot sole of right and left, or thyroid part.

By the thermal stimulus method, a level of the thermal stimulus can be evaluated. The rate of increase in blood flow volume in before and after the application of thermal stimulus is used as indicator of the application of thermal stimulus. From the effect of the thermal stimulus, the rate of increase in blood flow volume is desirably controlled to 60% or greater, preferably 100% or greater.

The action, in which blood vessels dilate and then blood flow increases, is considered to raise the production of calcitonin gene-related peptide (CGRP) with stimulus. It is considered that the release of CGRP and the like is accelerated by an increase in the blood flow of muscles since nerves, etc., are stimulated. That is, it is considered that, accordingly, the mechanism of axon reflex is activated with the stimulus of an afferent nerve or the like, thus releasing CGRP from terminal nerves and then dilating blood vessels in a dominant region.

Further, it is assumed that this also affects the autonomic nerve activity that is dominant in blood vessels. Blood vessels of muscles or the like are dominated by adrenergic sympathetic and cholinergic sympathetic nerves. The former is a vasoconstrictor nerve through an α-receptor, the latter is a vasodilator nerve through acetylcholine. The former is regularly active in a tense period to the blood vessels of muscles even during a resting time, and regularly maintains blood vessels in a constricted condition to some degree. On the other hand, the latter is considered to increase blood flow as it reacts to acetylcholine released from nerve endings and relaxes smooth muscles of blood vessels.

Thermal stimulus maintains the predominance of a parasympathetic nerve, which improves the acceleration state of sympathetic nerve over a long period. It is also considered that, due to an increase in a deep body temperature, blood pressure fluctuations and the like, thermal stimulus adjusts blood flow of each organ through systemic blood pressure and an autonomic nerve. With stimulus to a stimulus application site on a body surface according to the present invention, the effect on the mechanism and a sympathetic nerve becomes clear due to an increase in blood flow caused by the dilation of blood vessels.

Described is the mechanism of increasing blood flow with the application of stimulus to the thermal stimulus applied site. Psychological stress to a living body stimulates a sympathetic nerve through a limbic system and a hypothalamic pituitary, constricting blood vessels and lowering microcirculation of organs and else of a living body.

Moreover, as a bioactive hormone, vasoactive intestinal peptide (VIP) is secreted from a digestive tract, the pancreas and a hypothalamus, thus accelerating the peristaltic motion of intestines and increasing blood flow of a digestive tract. Additionally, due to a vascular endothelial growth factor (VEGF), vascularization and the vascular permeability of microvessels are accelerated. These VIP and VEGF significantly increase with stimulus to a specific site, thus it is considered that the blood flow of organs and peripheries is increased by triggering both functions of VIP and VEGF.

According to these results, it is considered that sympathetic nerve activity through an autonomic nerve and the over response of hypothalamic pituitary tropic hormone due to stress stimuli of a living body acted on the hypothalamus, suppressed the release of stress hormone and also increased blood flow through VIP and VEGF.

It is considered that, the blood flow is increased as reduction in the speed of blood flow through sympathetic flow response (SFR) due to stress suppressed the stress hormone and also increased blood flow through VIP and VEGF.

### Embodiment

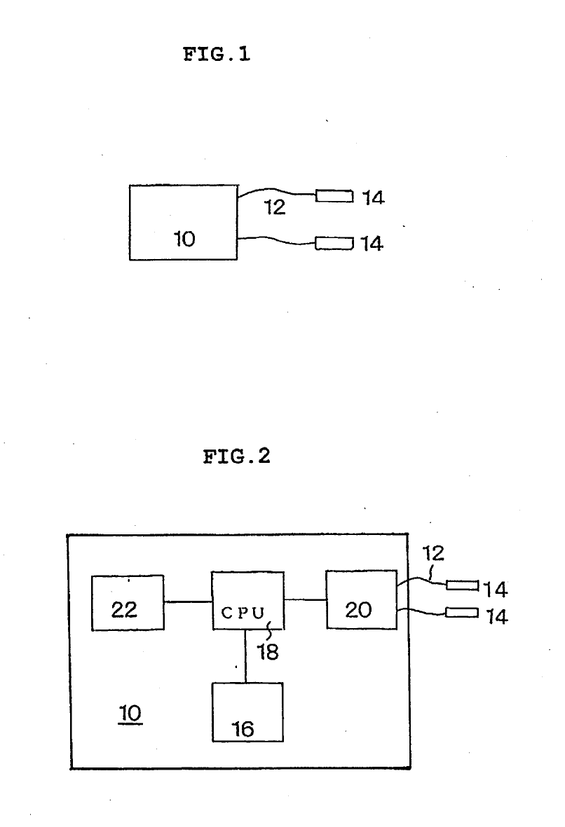

FIG. 1 is a schematic view of an electrical thermal stimulus apparatus according to the present invention.

FIG. 2 is a schematic view of a circuit diagram of the electrical thermal stimulus apparatus.

An electrical thermal stimulus apparatus comprises the apparatus 10, and thermal guide element 14 for a thermal stimulus, which is connected to the apparatus 10 by a lead 12.

As shown, the electrical thermal stimulus apparatus comprises a apparatus body 10 with a memory unit 16, in which the thermal stimulus patterns are stored, a control unit (CPU) 18, which reads out a thermal stimulus pattern from the memory unit 16, and an output unit 20, which supplies the thermal stimulus pattern to the thermal stimulus guide element 14 for a thermal stimulus. A thermal stimulus is applied to a thermal stimulus applied site, according to the thermal stimulus pattern.

A control unit (CPU) 18 is connected to the memory unit 16. A thermal stimulus pattern for obtaining a stimulus condition equivalent to that obtained from combustion of moxa is stored in the memory unit 16. The control unit (CPU) 18 reads out the thermal stimulus pattern from the memory unit 16, controls an output to a thermal stimulus guide element(s) based on detection of a temperature sensor 22, and outputs the thermal stimulus pattern to the thermal stimulus guide element(s)

The apparatus body 10 is connected to two or more thermal stimulus guide elements for a thermal stimulus in order to supply the thermal stimulus pattern to at least two different a thermal stimulus applied site. In this manner, the thermal stimulus pattern is applied to the thermal stimulus applied site through the thermal guide elements for thermal stimulus.

The thermal stimulus applied sites of a body surface comprises at least two of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

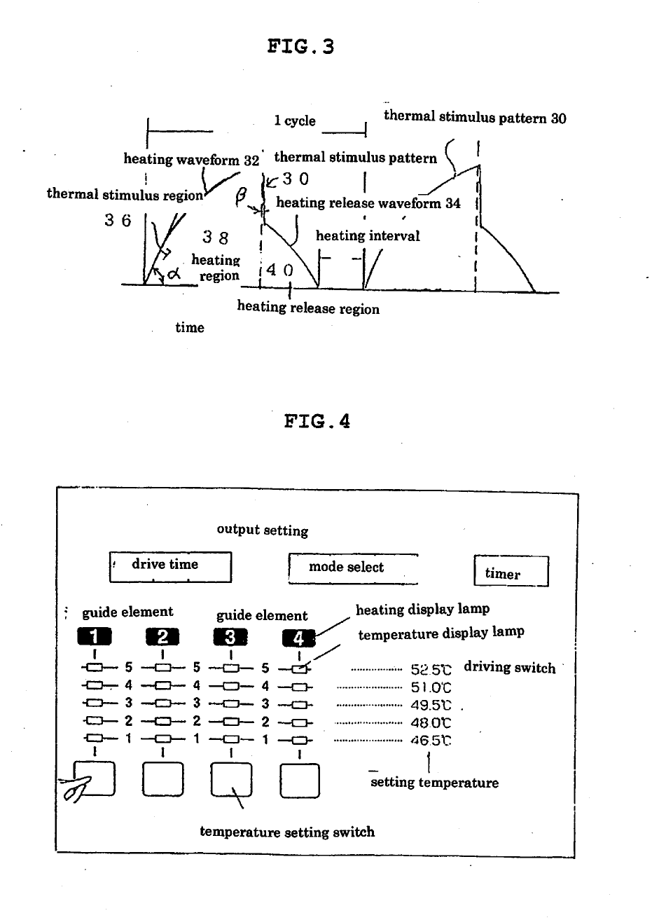

FIG. 3 shows a thermal stimulus waveform and cycle of a thermal stimulus waveform which is obtained by controlling a thermal stimulus apparatus.

An electrical thermal stimulus apparatus comprises a thermal stimulus control devices. The thermal stimulus control device controls at least one of thermal stimulus waveform, heating temperature, thermal stimulus strength, thermal applied time, thermal stimulus cycle, thermal stimulus pattern, warming mode, thermal stimulus protocol. The information from the thermal stimulus control device is output from a thermal guide element 14 for a thermal stimulus.

A electrical thermal stimulus apparatus applies thermal stimulus to thermal stimulus applied sites. And the volume of blood flow was measured before and after the application of stimulus to a central part of a horizontal line of a wrist joint using a laser Doppler tissue blood flowmeter. An increase in blood flow volume was recognized before and after the application of thermal stimulus. When the increase rate of blood flow is 60% or greater, preferably 100% or greater, the curative effect was remarkable.

The pattern of thermal stimulus desirably includes independent thermal stimulus waveforms whose phases are shifted so that the patterns of thermal stimulus do not substantially overlap each other.

The thermal stimulus waveform 30 includes a heating waveform 32 obtained by heating it to a predetermined peak temperature, for example, 50±5 degrees Celsius, and a thermal release waveform 34 which is formed by stopping heating after it reaches the peak temperature. A region surrounded by the heating waveform 32 and a thermal release waveform 34 is a thermal stimulus region 36. The thermal stimulus region provides a heating region 38 and a thermal release region 40.The thermal waveform 32 may be a convex shape heating waveform obtained by heating it to a predetermined peak temperature, for example, 50±5 degrees Celsius. Moreover, the thermal waveforms are not limited to the above-described thermal waveform. It may be a saw-toothed shape waveform, and concavo-convex waveform. Moreover, the heating waveform and the heat release waveform may be formed as a sine waveform.

A rising angle of the thermal waveform on the basis of the horizontal line is a,and a falling angle of the thermal waveform on the basis of the horizontal line of peak temperature is β. In the thermal stimulus release waveform, α is large than β.

The intensity of the thermal stimulus is obtained by varying the thermal stimulus. The intensity of the thermal stimulus is obtained by increasing an area of the thermal stimulus, by increasing a peak temperature, by increasing the gradient of the rising in the thermal stimulus waveform.

The thermal stimulus pattern comprises first thermal stimulus pattern 38 provided thermal stimulus region 36 comprising the heating waveform 32 and the thermal release waveform 34, and an interval between next thermal stimulus region 36, and second thermal stimulus pattern 40 formed by shifting the phase from first thermal stimulus pattern 38.

One cycle of the thermal stimulus pattern is desirably set so that a thermal stimulus region may be 10 second to 30 second and an interval time may be 1 second to 10 seconds. The pattern of thermal stimulus desirably includes independent thermal stimulus waveforms whose phases are shifted so that the patterns of thermal stimulus do not substantially overlap each other. The cycle of the thermal stimulus pattern is desirably repeated 1 [symbol] ˜10.

A temperature sensor is provided in a predetermined position of the housing which is in contact with a thermal stimulus applied site of a human body, and detects the temperature of the position, so as to send a detection signal to a sensor amplifier. The control unit (CPU) controls an output of an electric power generating circuit so that the temperature of the portion, which is in contact with a skin surface of a human body contact, may not exceed a predetermined temperature. In the warm temperature heating apparatus, the surface temperature of the guide elements for a thermal stimulus is controlled so as to be in a range from 40 to 50±5 degrees Celsius.

When the heating temperature of the thermal stimulus guide elements detected by the temperature sensor is equal to or lower than a reference temperature, a positive side period of a pulse signal is controlled so as to be long and a negative side period of the pulse signal is controlled so as to be short, according to the output of the temperature sensor. On the contrary, when it is in a state at the reference temperature, a positive side period is controlled so as to be short and a negative side period thereof is controlled so as to be long.

The apparatus body 10 is connected to two or more the thermal stimulus guide elements for a thermal stimulus in order to supply the thermal stimulus pattern to at least two different thermal stimulus applied sites In this manner, the thermal stimulus pattern is applied to the thermal stimulus applied site through the thermal stimulus guide elements for thermal stimulus.

A temperature sensor 22 is provided in a position(s) which is correlated with a temperature of an affected area near the heating elements. The thermal stimulus guide elements for a thermal stimulus have a structure set forth below. The thermal stimulus guide element 14 for a thermal stimulus comprises a casing of apparatus, a heater which is provided in the casing, and is used as a source of heating for applying thermal stimulus, a heat conduction board, which is provided on a lower face of the casing and which conducts heat of the heater to a skin of a patient, and a seal board provided on an upper face of the casing.

A temperature sensor is provided in a predetermined position of the housing which is in contact with a part of a human body, and detects the temperature of the position, so as to send a detection signal to a sensor amplifier. The control unit (CPU) controls an output of an electric power generating circuit so that the temperature of the thermal stimulus guide element which is in contact with a skin surface of a human body contact may not exceed a predetermined temperature. The surface temperature of the guide elements for a thermal stimulus is controlled so as to be in a range from 40 to 50±5 degrees Celsius.

When the heating temperature of the thermal stimulus guide elements detected by the temperature sensor is equal to or lower than a reference temperature, a positive side period of a pulse signal is controlled so as to be long and a negative side period of the pulse signal is controlled so as to be short, according to the output of the temperature sensor. On the contrary, when it is in a state at the reference temperature, a positive side period is controlled so as to be short and a negative side period thereof is controlled so as to be long.

The heat conduction plate of the thermal stimulus guide elements are made of at least two kinds of materials whose thermal conductivities(W/m-1/K-1) are different from each other. The thermal conductivities of the materials at approximately room temperature will be described below. At least two kind of materials whose thermal conductivities are different from each other are used, Carbon nonotube; 3000-5500, diamond; 1000-2000, silver; 420, cupper; 398, gold; 320, alminium; 236, silicon; 168, brass; 106, iron; 84, platinum; 70, stainless steel; 16.7-20, glass; 1, epoxy resin; 0.21, silicon rubber; 0.16.

Two kind of materials whose thermal conductivities are different from each other are used as follows; alumina and gold or copper or platinum or stainless steel or diamond, stainless steel and diamond or gold or platinum. Such a combination is not limited. In addition, for example, a combination of materials which are greatly different in electrical conductivity, is desirable, in case where a mental stress level is high.

Subjects were adult men and women. Burn injuries were taken into consideration so that an electrical thermal stimulus apparatus, MXA-8000 (SO-257), was used for a application of thermal stimulus. The thermal stimulus guide element has a diameter of 10 mm, and a heating temperature whose peak is 50±5 degrees Celsius was intermittently performed for 15 minutes. The thermal stimulus guide element was attached to the thermal stimulus applied site on a body surface, and the site was heated at 40 to 50 degrees Celsius.

A schematic diagram of a control panel for a thermal stimulus is performed by an electric thermal stimulus apparatus. Description of the case where the guide elements are fixed to two thermal stimulus applied sites, which are different from one another, will be given below. The guide elements are fixed to the above-mentioned selected thermal stimulus applied sites, and electric power is supplied from the electric thermal stimulus apparatus. Here, in temperature setting and stimulus application method of the thermal stimulus guide elements, when the number of thermal stimulus applied site is two, the thermal stimulus guide elements 1,and 2 are used. The temperature is adjusted by a temperature setting switch. Every time the switch is pushed, the temperature of the elements is controlled in a range of from 46.5 to 52.5° C. in order of 1-2.

A mode selection button for selecting a warming mode, that is, an alternate mode or a sequential mode, is provided therein. In the alternate mode, heating and pausing of the two guide elements 1 and 2 are alternatively repeated. In the sequential mode, thermal stimulus of the two guide elements which are different from one another is carried out one by one in order. In this way, thermal stimulus is non-simultaneously applied to the two different sites independently of one another. In addition, a mode of a heating interval (time) can be chosen. The alternate mode can be chosen. After, a interval mode (short, long) can be chosen. For example, in the alternate mode and short time, the guide elements 1 and 2 is heated alternately, and after five second (at time, after 10 second, also at long time, after 15 second), the heating may be stopped. In the sequential mode ,the guide elements 1 and 2 is heated for 7.5 second sequentially.

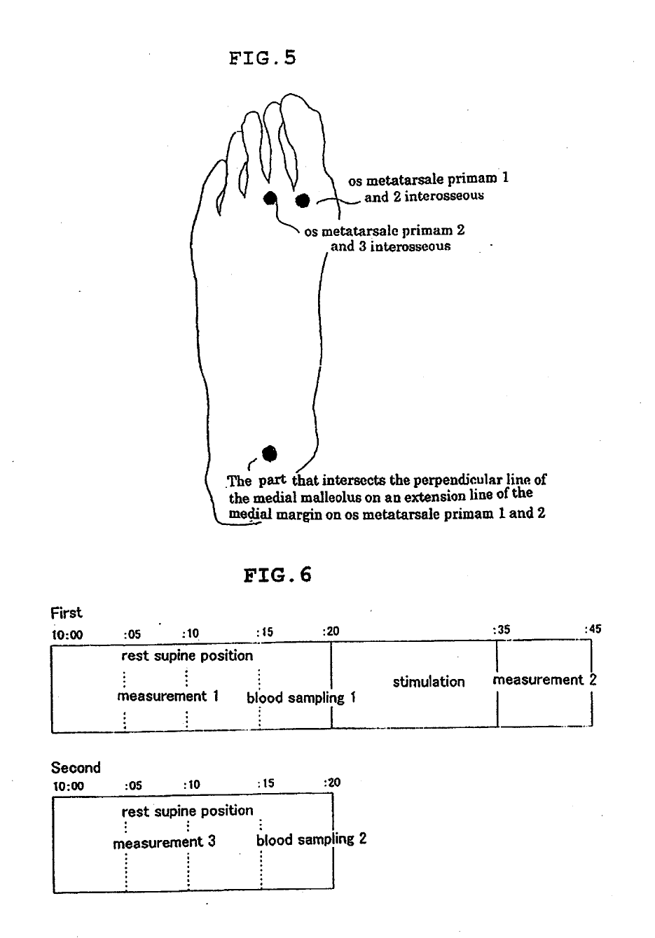

A thermal stimulus applied sites of a body surface comprises \textcircled{1}, part of os metatarsale primam 1 and 2 interosseous, \textcircled{2} part os metatarsale primam 2 and 3 interosseous, and \textcircled{3} part that intersects the perpendicular line of the medial malleolus on an extension line of the medial margin on os metatarsale primam 1 and 2 in foot sole of right and left (FIG. 5). And at these site, a thermal-stimulus is applied. At the thermal stimulus applied sire, a blood flow volume is increased and a deep body temperature increase.

A combination of a least two of thermal stimulus applied site is \textcircled{1} and \textcircled{2}, \textcircled{1} and \textcircled{3}, \textcircled{2} and \textcircled{3}, etc. And thermal stimulus applied site is \textcircled{1} or \textcircled{2} or \textcircled{3} at right and left foot. A thermal stimuli is applied to at least of two site asynchronous and independent of each other.

Taking into consideration influences on the psychological stress through autonomic nerves,

After keeping the subjects at rest in a state of a dorsal position for 20 minutes, a deep body temperature, a systolic blood pressure, salivary amylase, and heartrater(artery age) are measured. And thermal stimulus is performed for 15 minutes and they are measured again immediately after the thermal stimulus.

The protocol of thermal stimulus is shown in FIG. 6. At rest in a state of a dorsal position, Blood inspection, etc is performed, blood pressure is measured and blood is collected. After applied thermal stimulus, again Blood inspection, etc is performed, blood pressure is measured and blood is collected.

Table 1 shows a result of blood flow measured at before and after application of the thermal stimulus at a central area of a wrist joint horizontal line by using a laser Doppler tissue blood flow meter after allied the thermal stimulus at thermal stimulus applied site using the electrical thermal Stimulus apparatus. From the Table, the increase of blood flow is recognized in before and after application of the thermal stimulus. From Table, a strong therapeutic effect was observed in the rates of increase in blood flow are at 60% or more, or preferably 100% or more.

TABLE 1

| Patient | stimulation before | stimulation after | increase rate |
|---|---|---|---|
| No. 1 | 1.287 | 2.1036 | 63 |
| No. 2 | 3.2791 | 8.0098 | 144 |
| No. 3 | 1.7603 | 2.959 | 68 |
| No. 4 | 2.8101 | 4.6872 | 68 |
| No. 5 | 5.9857 | 13.0065 | 117 |
| No. 6 | 3.453 | 6.9631 | 102 |
| No. 7 | 2.0954 | 4.7401 | 126 |
| No. 8 | 1.4332 | 3.5227 | 146 |
| No. 9 | 1.0091 | 1.7377 | 72 |
| No. 10 | 3.9592 | 8.0699 | 104 |
| No. 11 | 1.5635 | 3.8743 | 148 |

Accordingly, there provided were an increase in a deep body temperature, a decline of systolic blood pressure, a decrease in salivary amylase, a decrease in cortisol, an improvement of liver functions, a decrease in total cholesterol, a decline of good and bad cholesterol, a decline of glucose level, a decline of AI (arteriosclerotic index) and a decrease in leptin. Furthermore, the balance of an autonomic nerve was measured with a heart rater, which showed an increase in resistance against stress.

The invention can apply a thermal stimulus therapy by increasing blood flow increases, for example therapy for release of psychological stress.

1. An electrical thermal stimulus apparatus comprising a thermal stimulus controller which controls at least one of a heating temperature, a thermal stimulus waveform, a thermal stimulus intensity, a thermal applied time, a cycle of a thermal stimulus waveform, a thermal stimulus pattern, a warming mode and a thermal stimulus protocol,
wherein the heating temperature is controlled at 46.5° C. to 52.5° C., and the thermal stimulus patterns are controlled independently by offsetting phases so as not to essentially overlap each other, and a blood flow before and after the application of thermal stimulus is measured with a laser Doppler tissue blood flowmeter mounted at the center of an inside wrist joint, and the rate of increase in blood flow after the application of thermal stimulus is 60% or more, or preferably 100 or more, the rate of increase in blood flow is used as an indicator of a stimulus evaluation.

2. The electrical thermal stimulus apparatus according to claim 1, wherein the thermal stimulus controller controls a cycle of thermal stimulus pattern comprising the thermal stimulus waveform configured by the heating waveform and a thermal release waveform, and an interval to the following thermal stimulus waveform, and in one cycle of the thermal stimulus patterns, a thermal stimulus waveform is set at 1 to 30 seconds and the interval between the thermal stimulus waveform is set at 1 to 10 seconds.

3. The electrical thermal stimulus apparatus according to claim 1, wherein the electrical thermal stimulus apparatus comprises an thermal stimulus guide element composed of two kinds of different metals.

4. A method for controlling a thermal stimulus controller of an electrical thermal stimulus apparatus,
wherein the thermal stimulus controller controls at least one of a heating temperature, a thermal stimulus waveform, a thermal stimulus intensity, a thermal applied time, a cycle of a thermal stimulus waveform, a thermal stimulus pattern, a warming mode and a thermal stimulus protocol,
wherein the heating temperature is controlled at 46.5° C. to 52.5° C. herein, and the thermal stimulus patterns are controlled independently by offsetting phases so as not to essentially overlap each other, and a blood flow before and after the application of thermal stimulus is measured with a laser Doppler tissue blood flowmeter mounted at the center of an inside wrist joint, and the rate of increase in blood flow after the application of thermal stimulus is 60% or more, or preferably 100% or more, the rate of increase in blood flow is controlled as an indicator for the stimulus application.

5. The method for controlling a thermal stimulus controller of an electrical thermal stimulus apparatus according to claim 4, wherein the thermal stimulus controller controls a cycle of thermal stimulus pattern comprising the thermal stimulus waveform configured by the heating waveform and a thermal release waveform, and an interval to the following thermal stimulus waveform, and in one cycle of the thermal stimulus patterns, a thermal stimulus waveform is set at 1 to 30 seconds and the interval between the thermal stimulus waveform is set at 1 to 10 seconds, and the thermal stimulus patterns are controlled independently by offsetting phases so as not to essentially overlap each other.

6. The method for controlling a thermal stimulus controller of an electrical thermal stimulus apparatus according to claim 5, wherein the thermal stimulus is applied the thermal stimulus applied site by thermal stimulus guide elements placed at a body surface comprising at least two of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

7. The method for controlling a thermal stimulus controller of an electrical thermal stimulus apparatus according to claim 4, wherein the thermal stimulus is applied the thermal stimulus applied site by thermal stimulus guide elements placed at a body surface comprising at least two of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

8. An electrical thermal stimulus apparatus comprising an electrical thermal stimulus controller, which controls non-simultaneously and independently applying a thermal stimulus with a peak temperature 50±5° C. by heating to thermal stimulus guide elements placed the different thermal stimulus applied sites of a body surface comprising the part that intersects the perpendicular line of the medial malleolus on extension line of the medial margin on os metatarsale primam 1 and 2 interosseous at foot of right and left to bring an increase rate of blood flow to 60% or more at before and after application of the thermal stimulus, wherein the blood flow is measured by a laser Doppler tissue blood flow meter attached to a central part of an inner side of a wrist joint and the increase rate of the blood flow is used as an indicator of a stimulus evaluation.
